(19) United States
(12) Patent Application Publication
(10) Pub. No.: US 2014/0323788 A1
(43) Pub. Date: Oct. 30, 2014

(54) PROCESS FOR MODIFYING AN APPARATUS AND FOR REMOVING ONE OR MORE CONTAMINANTS

(71) Applicant: UOP, LLC, Des Plaines, IL (US)

(72) Inventor: Dorothy M. Richmond, La Grange Park, IL (US)

(73) Assignee: UOP, LLC, Des Plaines, IL (US)

(21) Appl. No.: 13/869,736

(22) Filed: Apr. 24, 2013

(51) Int. Cl. C07C 7/12; C07C 7/00

(52) U.S. Cl. CPC C07C 7/12; C07C 7/005
USPC 585/802; 585/820; 29/401.1

(57) ABSTRACT

One exemplary embodiment can be a process for modifying an apparatus. The apparatus can include adding an adsorption zone upstream of a hydrocracking zone and a downstream of a vacuum distillation zone to adsorb polynuclear aromatic compounds originating from a feed provided to the vacuum distillation zone.

### FIELD OF THE INVENTION

This invention generally relates to modifying an apparatus and for removing one or more contaminants.

### DESCRIPTION OF THE RELATED ART

During operation of a vacuum column, a vacuum gas oil or a heavy vacuum gas oil can have entrained contaminants that may negatively impact the performance of a catalyst in a downstream hydrocracking unit. These contaminants may become entrained during upsets in or improper operation of the vacuum column. The types of contaminants may include polynuclear aromatics. Also, as many refineries are buying vacuum gas oils, such importation can raise the potential for significant loss of catalyst performance if the purchased vacuum gas oils contain coke precursors. Additionally other sources of feed such as cracked gas oils, a gas oil fraction of synthetic crudes, and deasphalted oils may contain coke precursors. Moreover, these contaminants may foul various parts of the refining equipment as they have a very low solubility level in a product hydrocarbon. They may tend to accumulate on the cold surfaces of heat exchangers used to recover heat from the effluent of a hydrocracking reactor. The coating caused by these deposits can decrease the efficiency of the heat recovery step and may lead to undesirably high pressure drops within the heat exchanger. At an extreme, the deposits may require termination of the processing in order to clean the heat exchangers. Hence, there is a desire to remove such contaminants from a feed entering the hydrocracking unit.

### SUMMARY OF THE INVENTION

One exemplary embodiment can be a process for modifying an apparatus. The apparatus can include adding an adsorption zone upstream of a hydrocracking zone and a downstream of a vacuum distillation zone to adsorb polynuclear aromatic compounds originating from a feed provided to the vacuum distillation zone.

Another exemplary embodiment may be a process for adsorbing polynuclear aromatic compounds. The process can include improperly operating a vacuum distillation column to produce a heavy vacuum gas oil stream, adsorbing the polynuclear aromatic compounds in an adsorption zone to minimize fouling of downstream equipment, and passing the heavy vacuum gas oil stream to a hydrocracking zone. Often, the heavy vacuum gas oil stream includes polynuclear aromatic compounds.

A further embodiment can be a process for removing one or more contaminants from a vacuum gas oil stream obtained from a vacuum distillation column experiencing non-optimum operation. The process can include sending the vacuum gas oil stream directly from a vacuum distillation zone to an adsorption zone. Often, the adsorption zone removes the contaminants.

The embodiments disclosed herein can provide an adsorption zone upstream of a hydrocracking zone. Desirably, the adsorption zone adsorbs contaminants such as coke precursors. The coke precursors can include polynuclear aromatic compounds. Particularly, installing such an adsorption zone upstream of a hydrocracking zone can remove contaminants originating from improper operation or operational upsets of a vacuum distillation column.

### DEFINITIONS

As used herein, the term “stream” can include various hydrocarbon molecules, such as straight-chain, branched, or cyclic alkanes, alkenes, alkadienes, and alkynes, and optionally other substances, such as gases, e.g., hydrogen, or impurities, such as heavy metals, and sulfur and nitrogen compounds. The stream can also include aromatic and nonaromatic hydrocarbons. Moreover, the hydrocarbon molecules may be abbreviated C1, C2, C3 . . . Cn where “n” represents the number of carbon atoms in the one or more hydrocarbon molecules. Furthermore, a superscript “+” or “−” may be used with an abbreviated one or more hydrocarbons notation, e.g., C3⁺or C3⁻¹, which is inclusive of the abbreviated one or more hydrocarbons. As an example, the abbreviation “C3⁺” means one or more hydrocarbon molecules of three carbon atoms and/or more. A “stream” may also be or include substances, e.g., fluids, other than hydrocarbons, such as hydrogen.

As used herein, the term “zone” can refer to an area including one or more equipment items and/or one or more sub-zones. Equipment items can include one or more reactors or reactor vessels, heaters, exchangers, pipes, pumps, compressors, and controllers.

Additionally, an equipment item, such as a reactor, dryer, or vessel, can further include one or more zones or sub-zones.

As used herein, the term “hydroprocessing” can refer to processing one or more hydrocarbons in the presence of hydrogen, and can include hydrotreating and/or hydrocracking.

As used herein, the term “hydrocracking” can refer to a process breaking or cracking bonds of at least one long chain hydrocarbon in the presence of hydrogen and at least one catalyst into lower molecular weight hydrocarbons.

As used herein, the term “polynuclear aromatics” can refer to the heavy aromatic hydrocarbons having six or more “benzene rings” and can be present in the heavier fractions of a crude oil. Often, these materials may concentrate in a bottoms stream, such as a vacuum residue, from a vacuum distillation column. During improper operation or upsets of the vacuum distillation column, sometimes these compounds may pass downstream to a hydrocracking reactor. Additionally, the term “polynuclear aromatics” may be abbreviated herein as “PNAs”.

As used herein, the term “directly” can mean communicating a stream without reacting, such as conducting a reaction with at least one other compound, or purifying with a process, such as flashing, adsorbing, or extracting. However, a stream can be communicated directly if it undergoes heating or cooling through, e.g., an exchanger.

As used herein, the term “vacuum gas oil” can include one or more C22-C52 hydrocarbons and boil in the range of about 340-about 590° C. at about 101.325 KPa. A vacuum gas oil may be a hydrocarbon product of vacuum distillation and be abbreviated herein as “VGO”.

As used herein, the term “heavy vacuum gas oil” can include one or more C36-C52 hydrocarbons and boil in the range of about 490-about 590° C. at about 101.325 KPa.

The term “heavy vacuum gas oil” may be abbreviated herein as “HVGO”.

As used herein, the term “medium vacuum gas oil” can include one or more C26-C36 hydrocarbons and boil in the range of about 400-about 490° C. at about 101.325 KPa. The term “medium vacuum gas oil” may be abbreviated herein as “MVGO”.

As used herein, the term “light vacuum gas oil” can include one or more C24-C26 hydrocarbons and boil in the range of about 370-about 400° C. at about 101.325 KPa. The term “light vacuum gas oil” may be abbreviated herein as “LVGO”.

As used herein, the terms “adsorbent” and “adsorber” may include, respectively, an absorbent and an absorber, and relates, but is not limited to, adsorption and/or absorption.

As used herein, the term “overhead stream” can mean a stream withdrawn at or near a top of a column, typically a distillation column.

As used herein, the term “bottom stream” can mean a stream withdrawn at or near a bottom of a column, typically a distillation column.

As used herein, the term “kilopascal” may be abbreviated “KPa”, the terms “cubic centimeter” may be abbreviated “cc”, the term “hour” may be abbreviated “hr”, and the terms “degrees Celsius” may be abbreviated “° C.”.

As depicted, the process flow lines in the figures can be referred to interchangeably as, e.g., lines, pipes, feeds, branches, portions, products, or streams.

### BRIEF DESCRIPTION OF THE DRAWINGS

FIG. 1 is a schematic, cross-sectional depiction of an exemplary apparatus.

FIG. 2 is a schematic, cross-sectional depiction of an exemplary adsorption zone.

### DETAILED DESCRIPTION

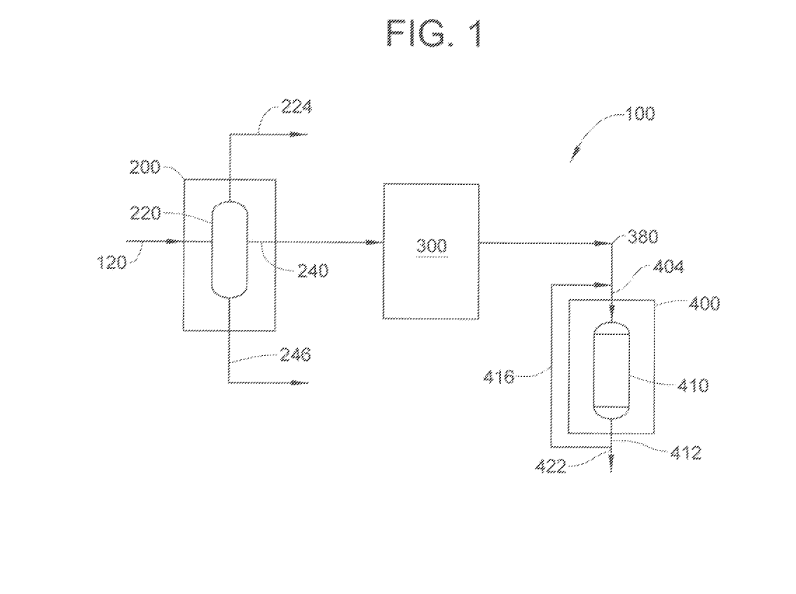

Referring to FIG. 1, an exemplary apparatus 100 can include a vacuum distillation zone 200, an adsorption zone 300, and a hydrocracking zone 400. The apparatus 100 may receive a feed 120 including an atmospheric bottom stream, typically obtained from an atmospheric distillation column processing a crude oil. Usually, the atmospheric bottom stream is an atmospheric gas oil having a boiling point range of about 200-about 380° C.

The vacuum distillation zone 200 can include a vacuum distillation column 220 and provide several streams, such as an overhead stream 224, a vacuum gas oil stream 240, and a bottom stream 246. Other streams may also be provided, such as a diesel cut, and the vacuum gas oil stream 240 can be provided as several streams, namely LVGO, MVGO, and HVGO streams. In one exemplary embodiment, the vacuum gas oil stream 240 may be an HVGO stream. Generally, the vacuum distillation column 220 is operated at a subatmospheric pressure. An exemplary vacuum distillation zone is disclosed in, e.g., U.S. Pat. No. 4,087,354. The heated VGO stream 240 may have a temperature of about 260-about 380° C. and a mildly superatmospheric pressure, such as a pressure of no more than about 360 KPa. Generally, the VGO stream 240 is sent directly to the adsorption zone 300.

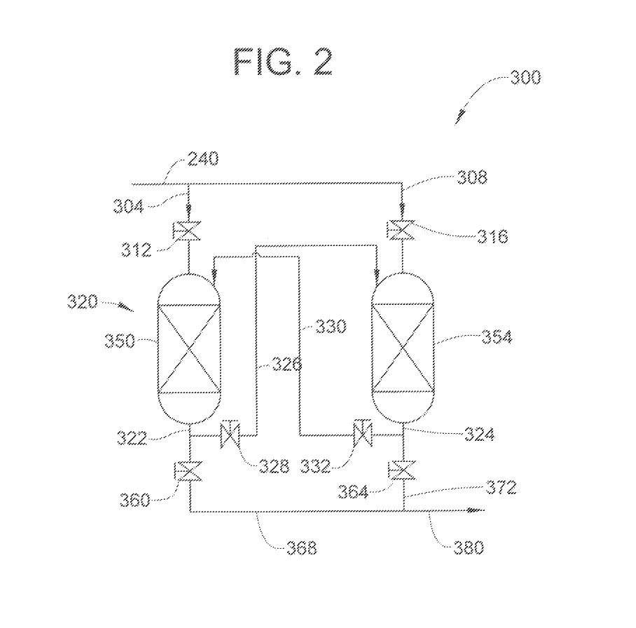

Referring to FIG. 2, the adsorption zone 300 can include several lines 304, 308, 322, 324, 326, 330, 368, and 372; valves 312, 316, 328, 332, 360, and 364; and a plurality of beds 320. The plurality of beds 320 can include a first bed 350 and a second bed 354. Each bed 350 and 354 may, independently, include a fixed bed of a small diameter particulate adsorbent, preferably inert. The adsorbent can include at least one of a silica gel, an activated carbon, an activated alumina, a silica-alumina gel, a clay, and a molecular sieve. Preferably, the adsorbent is rich in carbon. Charcoals can therefore be comprised in a preferred adsorbent. Desirably, the charcoal is substantially free of metals and may be derived from coconuts or other low metal content organic material. Exemplary adsorbents are disclosed in, e.g., U.S. Pat. No. 4,775,460. Often, an activated carbon adsorbent, e.g., activated charcoal, typically results in the selective separation of the PNAs from the VGO boiling range hydrocarbons and the accumulation of these polycyclic compounds on the activated carbon. The adsorption zone 300 can be operated at a pressure of about 340-about 3,500 KPa, and at a temperature of about 120-about 320° C., preferably at least about 260° C. Desirably, the space velocity for the adsorption zone 300 can be about 0.5-about 2.5 hr⁻¹.

In one exemplary embodiment, the first bed 350 and the second bed 354 can be operated in lead-lag mode. Particularly, the VGO stream 240 can pass first through the first bed 350 and then the second bed 354 by having the valves 312, 328, and 364 open and the valves 316, 332, and 360 closed. Isolating the second bed 354 can allow the replacement of adsorbent while the first bed 350 remains on-stream. Afterwards, the VGO stream 240 can pass first through the second bed 354 and then the first bed 350 by having the valves 316, 332, and 360 open and the valves 312, 328 and 364 closed. Changing the adsorbent in the first bed 350 can require first isolating the first bed 350 and solely using the second bed 354. Once changed, the first bed 350 can take the lead position, as described above.

Referring back to FIG. 1, a feed 380 for the hydrocracking zone 400 may exit the adsorption zone 300 and be combined with a recycle stream 416, as hereinafter described, to form a total feed stream 404 provided to the hydrocracking zone 400. The hydrocracking zone 400 can include at least one hydrocracking reactor 410 that may include one or more beds. The hydrocracking reactor 410 may be preceded by a hydrotreating reactor (not shown). The at least one hydrocracking reactor 410 can contain an acidic hydrocracking catalyst. The catalyst composition can also include a porous refractory inorganic oxide matrix which may form about 2-about 98%, by weight, of the support of the finished catalyst composite. The matrix may include any known refractory inorganic oxide such as alumina, magnesia, silica, titania, zirconia, silica-alumina, or a combination thereof. One preferred matrix may be silica-alumina or alumina The matrix can be produced by acid-treating, co-precipitation or successive precipitation from hydrosols. Optionally, this technique is combined with one or more activating treatments including hot oil aging, steaming, drying, oxidizing, reducing, and/or calcining. The pore structure of the support or carrier commonly defined in terms of surface area, pore diameter and pore volume, may be developed to specified limits by any suitable means including aging a hydrosol and/or hydrogel under controlled acidic or basic conditions at ambient or elevated temperature, or by gelling the carrier at a critical pH or by treating the carrier with various inorganic or organic reagents.

A finished catalyst for utilization in the hydrocracking reactor 410 should have a surface area of about 200-about 700 square meters per gram, a pore diameter of about 20-about 300 Angstroms, a pore volume of about 0.10-about 0.80 milliliters per gram, and apparent bulk density of about 0.50-about 0.90 gram/cc.

The catalyst may exist in the form of pills, pellets, granules, broken fragments, spheres, or various special shapes such as trilobal extrudates, disposed as a fixed bed within a reaction zone. Alternatively, the catalyst may be prepared in a suitable form for use in moving bed reaction zones in which the hydrocarbon charge stock and catalyst are passed either in countercurrent flow or in co-current flow, or in fluidized-solid processes in which the charge stock may be passed upward through a turbulent bed of finely divided catalyst, or in the suspension process, in which the catalyst is slurried in the charge stock and the resulting mixture is conveyed into the reaction zone. The charge stock may be passed through the reactor(s) in the liquid or mixed phase and in either upward or downward flow.

The catalyst particles may be prepared by any suitable method, such as the well-known oil drop and extrusion methods. In the case of the oil drop method, catalyst particles may be prepared by first suspending the selected zeolite powder in a suitable sol. Active metal components may also be incorporated into the sol. The sol admixture may then be passed as droplets into an oil bath which is maintained at an elevated temperature and retained in the oil bath until the sol droplets set to gelled spheres. The spherical particles may then be withdrawn from the oil bath and thereafter aged in a suspending medium at an elevated temperature for a suitable time period. The spherical particles may then be dried and calcined.

Although the hydrogenation components may be added before or during the oil drop or extrusion methods, hydrogenation components are preferably composited with the catalyst by impregnation after the selected zeolite and refractory inorganic oxide materials have been formed, dried and calcined. Impregnation of the metal hydrogenation component into the particles may be carried out in any manner known in the art including evaporative, dip and vacuum impregnation techniques. In general, the dried and calcined particles are contacted with one or more solutions which contain the desired hydrogenation components in dissolved form. After a suitable contact time, the composite particles can be dried and calcined to produce finished catalyst particles.

Hydrogenation components contemplated are those catalytically active metals selected from groups 6 and 8-10 of the periodic table. Generally, the amount of hydrogenation components present in the final catalyst composition is small compared to the quantity of the other above-mentioned components combined therewith. The metal from groups 8-10 generally comprises about 0.1-about 30%, by weight, preferably about 1-about 15%, by weight, of the final catalytic composite calculated on an elemental basis. The metal of group 6 comprises about 0.05-about 30%, by weight, preferably about 0.5-about 15%, by weight, of the final catalytic composite calculated on an elemental basis. As such, the hydrogenation component may include one or more metals of molybdenum, tungsten, chromium, iron, cobalt, nickel, platinum, palladium, iridium, osmium, rhodium, and rudinium.

The hydrogenation components are often present in the oxide form after calcination in air and may be converted to the sulfide form, if desired, by contact at elevated temperatures with a reducing atmosphere including hydrogen sulfide, a mercaptan or other sulfur containing compound. When desired, a phosphorus component may also be incorporated into the catalyst. Usually, phosphorus is present in the catalyst in the range of about 1-about 30%, by weight, calculated as P₂O₅. In addition, boron may also be present in the catalytic composite. An exemplary hydrocracking catalyst is disclosed in, e.g., U.S. Pat. No. 4,775,460.

The hydrocracking zone 400, including the hydrocracking reactor 410, may be useful in the production of middle distillate fractions boiling in the range of about 140-about 380° C., such as diesel fuels. Hydrogen may be added to the hydrocracking zone 400 to facilitate hydroprocessing. A hydrocracking reaction temperature can be about 180-about 650° C., preferably about 180-about 500° C., and reaction pressure can be up to about 25,000 KPa, preferably about 700-about 21,000 KPa. Contact times usually correspond to liquid hourly space velocities of about 0.1-about 15 hr⁻¹. Hydrogen circulation rates may be about 170-about 9,000 standard meter-cubed of hydrogen per meter-cubed of charge. Suitable conditions are disclosed in, e.g., U.S. Pat. No. 4,775,460.

An effluent stream 412 can exit the hydrocracking reactor 410 with a portion being separated as a recycle stream 416. The remainder can be obtained as a product stream 422 and sent to any suitable destination, such as the diesel pool. The recycle stream 416 may be separated from the effluent stream 412 in a fractionation unit (not shown) for which other product streams, such as the product stream 422, may be recovered.

The embodiments disclosed herein can be used to remove PNAs from a charge to a hydrocracking zone. Particularly, an adsorption zone can be installed upstream of a hydrocracking zone and downstream from a vacuum distillation zone. Hence, increased content of PNAs present in the feed, such as a VGO or HVGO, to the hydrocracking zone due to, e.g., improper operation of the vacuum distillation zone can be removed, and hence, avoid maintenance issues associated with PNAs plugging equipment such as coolers and exchangers.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing, all temperatures are set forth in degrees Celsius and, all parts and percentages are by weight, unless otherwise indicated.

From the foregoing description, one skilled in the art can easily ascertain the essential characteristics of this invention and, without departing from the spirit and scope thereof, can make various changes and modifications of the invention to adapt it to various usages and conditions.

1. A process for modifying an apparatus, comprising:
adding an adsorption zone upstream of a hydrocracking zone and a downstream of a vacuum distillation zone to adsorb polynuclear aromatic compounds originating from a feed provided to the vacuum distillation zone.

2. The process according to claim 1, wherein a vacuum gas oil is obtained from the vacuum distillation zone and comprises polynuclear aromatic compounds and further comprising passing the vacuum gas oil through the adsorption zone.

3. The process according to claim 2, further comprising improperly operating the vacuum distillation zone increasing the content of polynuclear aromatic compounds in a vacuum gas oil.

4. The process according to claim 1, wherein a heavy vacuum gas oil is obtained from the vacuum distillation zone and comprises polynuclear aromatic compounds and further passing the heavy vacuum gas oil through the adsorption zone.

5. The process according to claim 1, wherein the adsorption zone comprises a plurality of beds.

6. The process according to claim 5, further comprising operating the plurality of beds in a lead-lag mode.

7. The process according to claim 5, wherein the plurality of beds contains an adsorbent, in turn, comprising at least one of a silica gel, an activated carbon, an activated alumina, a silica-alumina gel, a clay, and a molecular sieve.

8. The process according to claim 1, wherein the hydrocracking zone comprises a hydrocracking reactor.

9. The process according to claim 8, wherein the hydrocracking reactor contains a catalyst comprising at least one metal of groups 6 and 8-10 of the periodic table and a matrix.

10. The process according to claim 9, wherein the matrix comprises at least one of an alumina, a magnesia, a silica, a titania, a zirconia, and a silica-alumina.

11. The process according to claim 9, wherein the hydrocracking reactor operates at a temperature of about 180-about 500° C. and a pressure of about 700-about 21,000 KPa.

12. The process according to claim 1, wherein the feed comprises an atmospheric bottom stream.

13. A process for adsorbing polynuclear aromatic compounds, comprising:
A) improperly operating a vacuum distillation column to produce a heavy vacuum gas oil stream wherein the heavy vacuum gas oil stream comprises polynuclear aromatic compounds;
B) adsorbing the polynuclear aromatic compounds in an adsorption zone to minimize fouling of downstream equipment; and
C) passing the heavy vacuum gas oil stream to a hydrocracking zone.

14. The process according to claim 13, wherein the adsorption zone comprises a plurality of beds.

15. The process according to claim 14, wherein the plurality of beds contains an adsorbent, in turn, comprising at least one of a silica gel, an activated carbon, an activated alumina, a silica-alumina gel, a clay, and a molecular sieve.

16. The process according to claim 13, wherein the hydrocracking reactor contains a catalyst comprising at least one metal of groups 6 and 8-10 of the periodic table and a matrix.

17. The process according to claim 16, wherein the matrix comprises at least one of an alumina, a magnesia, a silica, a titania, a zirconia, and a silica-alumina.

18. The process according to claim 16, wherein the hydrocracking reactor operates at a temperature of about 180-about 500° C. and a pressure of about 700-about 21,000 KPa.

19. A process for removing one or more contaminants from a vacuum gas oil stream obtained from a vacuum distillation column experiencing non-optimum operation, comprising:
sending the vacuum gas oil stream directly from a vacuum distillation zone to an adsorption zone wherein the adsorption zone removes the contaminants.

20. The process according to claim 19, wherein the adsorption zone contains at least one of a silica gel, an activated carbon, an activated alumina, a silica-alumina gel, a clay, and a molecular sieve for removing one or more polynuclear aromatic compounds and the contaminants comprise one or more polynuclear aromatic compounds.
